(19) United States
(12) Patent Application Publication
(10) Pub. No.: US 2009/0029357 A1
(43) Pub. Date: Jan. 29, 2009

(54) Method for diagnosing cancer using cancer-associated deletion gene marker

(75) Inventors: Johji Inazawa, Tokyo (JP); Issei Imoto, Tokyo (JP); Hiroyuki Izumi, Tokyo (JP); Sana Yokoi, Tokyo (JP)

Correspondence Address: BIRCH STEWART KOLASCH & BIRCH, PO BOX 747, FALLS CHURCH, VA, 22040-0747, US

(73) Assignees: FUJIFILM Corporation, Tokyo (JP); Tokyo Medical and Dental University, Tokyo (JP)

(21) Appl. No.: 11/783,723

(22) Filed: Apr. 11, 2007

(30) Foreign Application Priority Data
Apr. 12, 2006 (JP) 2006-109312

(51) Int. Cl. C12Q 1/68; G01N 33/53

(52) U.S. Cl. 435/6; 436/501

(57) ABSTRACT

It is an object of the present invention to provide a novel method for diagnosing cancer by discovering a cancer-associated gene which could not be detected by a conventional technique, and detecting deletion, mutation, or an abnormal expression level of such cancer-associated gene. The present invention provides a method for diagnosing cancer which comprises a step of detecting deletion of the GMDS, ANKRD15, TEK, or EBI2 gene in a test sample by using DNA containing all or part of said gene.

### TECHNICAL FIELD

The present invention relates to a method for diagnosing cancer by analyzing or detecting a cancer-associated gene or a product thereof in a specimen.

### BACKGROUND ART

It has been known that onset of cancer is induced by mutation or quantitative change of a cell protein. Along with recent development in genetic engineering, it has become possible to amplify a gene encoding a specific protein and to analyze gene mutation in cancer cells, resulting in breakthroughs in the field of cancer research. Hitherto, analysis and identification of oncogenes involved in the oncogenic transformation of cells and the abnormal growth of cancer cells have made progress. Meanwhile, in recent years, cancer-suppressing genes have been gaining attention. Mutation or the decreased expression level of cancer-suppressing gene leads to oncogenic transformation of cells. Examples of cancer-suppressing genes that have been identified include Rb gene of retinoblastoma, p53 gene and APC gene of large-bowel cancer, and WT1 gene of Wilms tumor. For instance, an example of a cancer-suppressing agent that uses WT1 gene has been reported (WO2003/002142).

In addition, it has been gradually revealed that cancer development, malignant progression, and metastasis are caused by abnormalities of not only a single gene but also a plurality of genes. In addition, a greater number of unidentified oncogenes and cancer-suppressing genes are now believed to exist. There are many genes known to have effects that suppress cancer. In most cases, screening for such genes has been carried out by an approach of visually detecting mutation of a patient's gene via staining of chromosomal DNA (Yasuhide Yamashita, et al., World J Gastroenterol, 11 (33): 5129-5135, 2005) or by a method wherein a region of gene deletion is roughly selected based on LOH (loss of heterozygosity) analysis so that important gene regions are narrowed down (WO01/032859). However, such methods are not sufficient as means of discovering cancer-suppressing genes. This is because a tremendous number of DNA deletion regions are detected, so that narrowing them down into important gene regions is extremely time- and labor-consuming, which has been a drawback. Further, conventional separation and discrimination methods for pathological conditions of cancer have only been able to determine malignancy with difficulty.

### DISCLOSURE OF THE INVENTION

It is an object of the present invention to provide a novel method for diagnosing cancer by discovering a cancer-associated gene which could not be detected by a conventional technique, and detecting deletion, mutation, or an abnormal expression level of such cancer-associated gene.

To achieve the above objects, the present inventors have intensively searched for partially deleted DNA regions in non-small cell lung cancer cases and identified genes differing in methylation degree. Non-small cell lung cancer is further classified by tissue type, including adenocarcinoma, planocellular carcinoma, large cell carcinoma, squamous cell carcinoma, and the like, and it accounts for 80% or more of lung cancer cases. In order to specify deleted DNA in non-small cell lung cancer, the present inventors have screened for genes deleted at high frequencies in cancer via a newly developed array CGH method (Inazawa J., et al., Cancer Sci. 95 (7), 559, 2004). As a result, the present inventors succeeded in isolating a gene that is homozygously deleted in genomic DNA, i.e., a gene that lacks genomic region in the non-small cell lung cancer cell line. Detection of the existence of such gene enabled cancer diagnosis. The present invention has been completed based on such finding.

The present invention provides a method for diagnosing cancer which comprises a step of detecting deletion of the GMDS, ANKRD15, TEK, or EBI2 gene in a test sample by using DNA containing all or part of said gene.

The present invention further provides a method for diagnosing cancer which comprises a step of analyzing the GMDS, ANKRD15, TEK, or EBI2 gene in a test sample by using DNA or RNA containing all or part of the GMDS, ANKRD15, TEK, or EBI2 gene.

Preferably, the analysis involves detection of mutation of the gene or detection of abnormal expression level of the gene.

The present invention provides a method for diagnosing cancer which comprises a step of analyzing GMDS, ANKRD15, TEK, or EBI2 protein in a test sample using an antibody against GMDS, ANKRD15, TEK, OR EBI2 protein or fragment thereof.

Preferably, the analysis involves detection of abnormal expression level of the protein.

Preferably in the method of the present invention, lung cancer is diagnosed.

### BEST MODE FOR CARRYING OUT THE INVENTION

Hereafter, the embodiments and implementation of the present invention will be described in detail.

The cancer-associated gene, which is targeted in the method for diagnosing cancer according to the present invention, is the GMDS gene, the ANKRD15 gene, the TEK gene, or the EBI2 gene.

The nucleotide sequences of the GMDS gene, the ANKRD15 gene, the TEK gene, and the EBI2 gene and the amino acid sequences of the GMDS protein, the ANKRD15 protein, the TEK protein, and the EBI2 protein are already known. Such sequence information is available from the database of the National Center for Biotechnology Information (NCBI).

### GMDS:

GMDS is known as GMD or GDP-mannose 4,6-dehydratase (EC 4.2.1.47) (GDP-D-mannose dehydratase), and it has the RefSeq ID of NM_{—}001500 (SEQ ID NO: 1). The amino acid sequence thereof is shown (SEQ ID NO: 2). In the human genome, it is contained in clone RP11-79M24 (available from the UCSC genome browser (http://genome.ucsc.edu/cgi-bin/hgGateway)).

### ANKRD15:

ANKRD15 is known as DKFZp451G231, KANK, KIAA0172, MGC43128, or the ankyrin repeat domain 15, and it has RefSeq IDs of NM_{—}015158 and NM_{—}153186 (SEQ ID NO: 3 and SEQ ID NO: 4). The amino acid sequences thereof are shown (SEQ ID NO: 5 and SEQ ID NO: 6). In the human-genome, they are contained in clone RP11-31F19. They are reported to function as cancer suppressor genes in renal cancer cells (J. Biol. Chem., Vol. 277, Issue 39, 36585-36591, 2002).

### TEK:

TEK is known as CD202B, TIE-2, VMCM (venous malformations, multiple cutaneous and mucosal), VMCM1, or TEK tyrosine kinase, endothelial, and it has the RefSeq ID of NM_{—}000459 (SEQ ID NO: 7). The amino acid sequence thereof is shown (SEQ ID NO: 8). In the human genome, it is contained in clone RP11-33015. It is known as an angiopoietin 1 receptor having tyrosine kinase activity, which is involved in vascular endothelial cell growth (Cell 118: 149-161, 2004).

### EBI2:

EBI2 is known as EBV-induced G-protein coupled receptor 2 or Epstein-Barr virus induced gene 2 (lymphocyte-specific G protein-coupled receptor), and it has the RefSeq ID of NM_{—}004951 (SEQ ID NO: 9). The amino acid sequence thereof is shown (SEQ ID NO: 10). In the human genome, it is contained in clone RP11-72J7. EBI2 is a G-protein-coupled receptor, the ligand of which has not been discovered, and it is known to be induced by infection with EB viruses (J. Virol. 67: 2209-2220, 1993).

Herein, the term “gene” refers to a human-derived gene (genome DNA or cDNA) that is specified with the above nucleotide sequence. The term “protein” refers to a protein that is specified with the above amino acid sequence and encoded by gene.

The GMDS, ANKRD15, TEK, or EBI2 gene may be cDNA obtained from cultured cells using a technique known by persons skilled in the art, or may be synthesized by PCR or the like based on the nucleotide sequences derived from clone information set forth in Table 1 of the present specification. When DNA having the nucleotide sequence set forth in SEQ ID NOs is obtained by PCR, PCR is carried out using a human chromosomal DNA or a cDNA library as a template and a pair of primers designed to be able to amplify the nucleotide sequence derived from clone information set forth in Table 1. DNA fragments amplified by PCR can be cloned into an adequate vector that can be amplified in a host such as Escherichia coli.

The aforementioned operations, such as probe or primer preparation, cDNA library construction, screening of a cDNA library, and cloning of a target gene, are known to persons skilled in the art. Such operations can be carried out in accordance with methods described in Molecular Cloning: A laboratory Manual, 2^{nd} Ed., Cold Spring Harbor Laboratory, Cold Spring Harbor, N.Y., 1989, Current Protocols in Molecular Biology, Supplement 1-38, John Wiley & Sons (1987-1997) and the like.

A homologous gene of GMDS, ANKRD15, TEK, or EBI2 gene may be used. In accordance with the present invention, the term “homologous gene” refers to: a gene having a nucleotide sequence encoding a cancer-related protein, such nucleotide sequence being derived from the nucleotide sequence derived from clone information set forth in Table 1 by deletion, addition, or substitution of one or several nucleotides; or a gene having a nucleotide sequence encoding a cancer-related protein, such nucleotide sequence being hybridized with the nucleotide sequence derived from clone information set forth in Table 1 under stringent conditions. In addition, a fragment of GMDS, ANKRD15, TEK, or EBI2 gene is included in the definition of homologous gene of GMDS, ANKRD15, TEK, or EBI2 gene.

Regarding the above “nucleotide sequence derived from the nucleotide sequence derived from clone information set forth in Table 1 by deletion, addition, or substitution of one or several nucleotides,” the range of “one to several amino acids” is not particularly limited. For instance, such description indicates 1 to 60 nucleotides, preferably 1 to 30 nucleotides, more preferably 1 to 20 nucleotides, further preferably 1 to 10 nucleotides, and particularly preferably 1 to 5 nucleotides.

Thus, as long as the “homologous gene of GMDS, ANKRD15, TEK, or EBI2 gene” has the structure and function described above, its origin is not particularly limited. Therefore, it may be derived from mammals excluding humans, or may be obtained by artificially introducing mutation into a gene derived from mammals such as humans.

The aforementioned “gene having a nucleotide sequence encoding a cancer-related protein, such nucleotide sequence being derived from the nucleotide sequence derived from clone information set forth in Table 1 by deletion, addition, or substitution of one or several nucleotides” can be produced by any methods known by persons skilled in the art such as chemical synthesis, gene engineering techniques, and mutagenesis methods. Specifically, the aforementioned gene can be obtained by utilizing DNA having the nucleotide sequence derived from clone information set forth in Table 1 and introducing mutation into the DNA. For instance, a method wherein DNA having the nucleotide sequence derived from clone information set forth in Table 1 is allowed to come into contact with an agent serving as a mutagen, a method of UV irradiation, a gene engineering technique, and the like can be used. Site-directed mutagenesis is one of gene engineering techniques. It is useful because a specific mutation can be introduced into a specific site. This technique can be carried out in accordance with, for example, Molecular Cloning, A laboratory Manual, 2^{nd} Ed., Cold Spring Harbor Laboratory, Cold Spring Harbor, N.Y., 1989; Current Protocols in Molecular Biology, Supplements 1-38, John Wiley & Sons (1987-1997).

The aforementioned “nucleotide sequence being hybridized under stringent conditions” refers to a nucleotide sequence of DNA obtained by colony hybridization, plaque hybridization, Southern hybridization, or the like using DNA as a probe. For instance, an example of the DNA used is DNA that can be identified by carrying out hybridization at 65° C. in the presence of 0.7 to 1.0 M NaCl using a filter in which DNA derived from a colony or plaque or a fragment thereof is immobilized, followed by washing of the filter at 65° C. using 0.1 to 2×SSC solution (1×SSC solution comprises 150 mM sodium chloride and 15 mM sodium citrate). Hybridization can be carried out in accordance with methods described in Molecular Cloning: A laboratory Manual, 2^{nd} Ed., Cold Spring Harbor Laboratory, Cold Spring Harbor, N.Y., 1989.

An example of DNA hybridized under stringent conditions is DNA having a certain level or more of homology to the nucleotide sequence of DNA used as a probe. For instance, such DNA has a homology of 70% or more, preferably 80% or more, more preferably 90% or more, further preferably 93% or more, and particularly preferably 95% or more to the DNA used as a probe.

The aforementioned “gene having a nucleotide sequence encoding a cancer-related protein, such nucleotide sequence being hybridized with the nucleotide sequence derived from clone information set forth in Table 1 under stringent conditions” can be obtained as described above by colony hybridization, plaque hybridization, or Southern hybridization under certain hybridization conditions.

In the present invention, DNA containing part of the GMDS, ANKRD15, TEK, or EBI2 gene may be used to analyze each gene in the test sample. The term “part of the GMDS, ANKRD15, TEK, or EBI2 gene” used herein refers to, for example, an oligonucleotide comprising a nucleotide sequence of about 10 to 30 consecutive nucleotides, among the nucleotide sequences derived from the clone information as shown in Table 1.

The detection for screening cancer can be carried out by analyzing GMDS, ANKRD15, TEK, or EBI2 gene in a test sample using DNA or RNA containing GMDS, ANKRD15, TEK, or EBI2 gene in its entirety or a part thereof as a primer or probe. Specifically, the expression “analyzing GMDS, ANKRD15, TEK, or EBI2 gene” used herein indicates detection of deletion of genome DNA, or detection of gene mutation or abnormal level of gene expression.

Detection of gene mutation is carried out as follows. When the above DNA or RNA is used as a primer, a partial sequence of DNA prepared from a test sample is amplified by PCR using, for example, selected two types of primers

Meanwhile, detection of abnormal levels of gene expression can be carried out by Northern hybridization or RT-PCR (reverse transcription-polymerase chain reaction) using a probe containing the above RNA sequence.

Examples of a test sample that can be used include tissue section, blood, lympha, sputum, lung lavage liquid, urine, feces, and tissue culture supernatant, which are suspected to contain the presence of tumor.

In the present invention, cancer can be diagnosed by analyzing the GMDS, ANKRD15, TEK, or EBI2 proteins in a test sample using an antibody against GMDS, ANKRD15, TEK, or EBI2 protein or a fragment thereof.

The antibody against the GMDS, ANKRD15, TEK, or EBI2 protein used in the present invention (hereafter referred to as the antibody) can be produced by a conventional method using the GMDS, ANKRD15, TEK, or EBI2 protein in its entirety or a part thereof as an antigen. The term “a part of an GMDS, ANKRD15, TEK, OR EBI2 protein” indicates a polypeptide comprising at least 6 amino acids, preferably about 8 to 10 amino acids, and further preferably about 11 to 20 amino acids, which are consecutive amino acids constituting the amino acid sequence of the GMDS, ANKRD15, TEK, OR EBI2 protein set forth in the Sequence Listing. The GMDS, ANKRD15, TEK, OR EBI2 protein in its entirety or a part thereof serving as an antigen may be prepared by either biological or chemical techniques.

A polyclonal antibody can be prepared in the following manner: the above antigen, for example, is repeatedly used for subcutaneous, intramuscular, intraperitoneal, and intravenous inoculation in animals such as mice, guinea pigs, and rabbits such that the animals are sufficiently immunized; blood is collected from the animals; and serum separation is performed. A monoclonal antibody can be prepared from a culture supernatant of a hybridoma or ascites of a mouse into which the hybridoma has been administered, such hybridoma being obtained by cell fusion of commercially available mouse myeloma cells and splenic cells of a mouse immunized with, for example, the above antigen.

It is possible to measure the expression level of GMDS, ANKRD15, TEK, or EBI2 protein in a test sample using an antibody against GMDS, ANKRD15, TEK, or EBI2 protein or a fragment thereof prepared as described above. For instance, the measurement of the expression level can be carried out using Western blotting or immunological methods such as immunoblotting, enzymeimmunoassay (EIA), radioimmunoassay (RIA), a fluorescent antibody method, and immunocyto-staining. Herein, a fragment of GMDS, ANKRD15, TEK, OR EBI2 protein antibody refers to a single-chain fragment variable (scFv) of an antibody of interest or the like.

Examples of a test sample that can be used include tissue section, blood, lympha, sputum, alveolar lavage liquid, urine, feces, and tissue culture supernatant, which are suspected to exhibit the presence of tumor. The low expression level of GMDS, ANKRD15, TEK, or EBI2 protein in a test sample subjected to measurement indicates that the expression of the GMDS, ANKRD15, TEK, or EBI2 gene is suppressed in the sample tissue or cells. Thus, cancer can be diagnosed.

Examples of cancer to be diagnosed in the method for diagnosing cancer according to the present invention include, but are not limited to, malignant melanoma, malignant lymphoma, lung cancer, esophageal cancer, gastric cancer, large-bowel cancer, rectal cancer, colon cancer, urinary tract tumor, gallbladder cancer, bile duct cancer, biliary tract, breast cancer, liver cancer, pancreatic cancer, testis tumor, maxillary cancer, tongue cancer, lip cancer, oral cavity cancer, pharynx cancer, larynx cancer, ovarian cancer, uterine cancer, prostate cancer, thyroid gland cancer, brain tumor, Kaposi's sarcoma, angioma, leukemia, polycythemia vera, neuroblastoma, retinoblastoma, myeloma, bladder tumor, sarcoma, osteosarcoma, myosarcoma, skin cancer, basal-cell carcinoma, cutaneous appendage tumor, skin metastatic cancer, and skin melanoma. Preferably, the cancer to be diagnosed is lung cancer, and particularly preferably non-small cell lung cancer.

The present invention is hereafter described in greater detail with reference to the following examples, but the technical scope of the present invention is not limited thereto.

### EXAMPLES

### (1) Experimental Materials

Cell lines used herein were squamous cell lines EBC-1, LK-2, PC10, VMRC-LCP, LC-1sq, and ACC-LC-73; adenocarcinoma cell lines 11-18, A549, ABC-1, RERF-LC-OK, VMRC-LCD, SK-LC-3, and RERF-LC-KJ; and large cell carcinoma cell lines KNS-62, 86-2, LU65, PC-13, ACC-LC-33, NCI-H460, and LU99A. As lung cancer samples derived from clinical specimens, paraffin-embedded samples derived from 53 types of adenocarcinoma and rapidly-frozen samples derived from 59 types of adjacent, normal site were used. Cancer samples derived from clinical specimens were obtained from the National Cancer Center, Hokushin General Hospital of Nagano Prefectural Welfare Federation and used under agreement with each patient and approval of the ethical committee of each organization. Moreover, clinical specimen donors were not subjected to radiation, chemical therapy, or immunological therapy before sampling.

### (2) Separation of Deleted DNA in Non-Small Cell Lung Cancer Cell by Array CGH

Non-small cell lung cancer cell lines were screened for a homozygously deleted gene via the array CGH method using an MCG Whole Genome Array-4500 (Inazawa J., et al., Cancer Sci. 95(7), 559, 2004). DNA derived from a cancer cell was labeled with Cy3 and then mixed with a healthy subject control sample labeled with Cy5, followed by hybridization. BAC clones (each exhibiting a logarithmic value of −2.0 or lower (with 2 as the lowest) obtained by dividing the fluorescent signal intensity of the former DNA by the same of the latter DNA) and the genes contained in such clones are listed in Table 1. Deleted regions were found in various cancer cells; that is, gene deletion represented by cancer-suppressing gene CDKN2 was detected. In the non-small cell lung cancer cell lines, GMDS (6p25: GDP-mannose 4,6-dehydratase), ANKRD15 (9p24.3: ankyrin repeat domain 15), TEK (9p21.3: TEK tyrosine kinase, endothelial, TIE2), and EBI2 (13q32.2: Epstein-Barr virus induced gene 2 (lymphocyte-specific G protein-coupled receptor)) were found to be deleted. It was suggested that such deleted gene would function as a cancer-associated gene in the non-small cell lung cancer cell, and it was demonstrated that such deletion could be used as a gene deletion marker for non-small cell lung cancer.

TABLE 1
BAC clones separated as homozygously deleted DNAs from non-small cell lung
cancer cells via array CGH, and genes contained in such regions

| | | Locus^{a} | | Cell line (Total 20) | | | Number of |
|---|---|---|---|---|---|---|---|
| No. | BAC | Chr. Band | Position | n | Name | Possible candidate gene^{b} | known genes |
| 1 | RP11-178M15 | 1p36.21 | chr1:13,731,635-13,731,966 | 1 | 11-18 | PRDM2 | 2 |
| 2 | RP11-79M24 | 6p25 | chr6:1,754,013-1,926,781 | 1 | KNS-62 | GMDS | 1 |
| 3 | RP11-31F19 | 9p24.3 | chr9:537,217-682,143 | 1 | LC-1 sq | ANKRD15 | 7 |
| | RP11-143M15 | 9p24.3 | chr9:812,146-991,152 | 1 | LC-1 sq | | |
| 4 | RP11-113D19 | 9p21.3 | chr9:20,996,400-21,158,464 | 1 | VMRC-LCD | CDKN2A, CDKN2B, MTAP | 22 |
| | | | | | VMRC-LCD, LC-1 sq. | | |
| | RP11-344A7 | 9p21.3 | chr9:21,506,373-21,676,227 | 4 | KNS-62 | | |
| | RP11-11J1 | 9p21.3 | chr9:22,417,726-22,579,721 | 1 | KNS-62 | | |
| | RP11-782K2 | 9p21.3 | chr9:22,584,981-22,585,358 | 1 | KNS-62 | | |
| 5 | RP11-33O15 | 9p21.3 | chr9:22,823,087-22,897,484 | 2 | KNS-62, LU99A | TEK | 6 |
| | RP11-330J23 | 9p21.3 | chr9:25,292,197-25,425,886 | 2 | KNS-62, LU99A | | |
| | RP11-55P9 | 9p21.3-21.2 | chr9:25,425,787-25,573,596 | 1 | LU99A | | |
| 6 | RP11-307D17 | 13q21.2 | chr13:58,727,654-61,368,564 | 1 | VMRC-LCD | PCDH2O | 2 |
| 7 | RP11-600P1 | 13q31.1 | chr13:77,973,420-78,566,321 | 1 | VMRC-LCP | | 4 |
| | RP11-25J23 | 13q31.1 | chr13:78,837,347-79,310,431 | 1 | VMRC-LCP | | |
| | RP11-440G4 | 13q31.1 | chr13:81,026,555-81,027,175 | 1 | VMRC-LCP | | |
| | RP11-295L12 | 13q31.1 | chr13:81,571,893-82,147,460 | 1 | VMRC-LCP | | |
| | RP11-400M8 | 13q31.1 | chr13:82,201,212-82,280,653 | 1 | VMRC-LCP | | |
| | RP11-91O15 | 13q31.1 | chr13:83,668,529-83,670,198 | 1 | VMRC-LCP | | |
| | RP11-1181K20 | 13q31.1 | chr13:83,711,618-83,853,235 | 1 | VMRC-LCP | | |
| | RP11-29C8 | 13q31.1 | chr13:84,853,779-85,313,978 | 1 | VMRC-LCP | | |
| | RP11-569120 | 13q31.1 | chr13:85,891,468-86,093,892 | 1 | VMRC-LCP | | |
| | RP11-29P20 | 13q31.2 | chr13:86,796,651-87,270,500 | 1 | VMRC-LCP | | |
| | RP11-27D9 | 13q31.2 | chr13:87,913,237-88,370,388 | 1 | VMRC-LCP | | |
| | RP11-114G1 | 13q31.3 | chr13:88,656,527-89,117,305 | 1 | VMRC-LCP | | |
| | RP11-86C3 | 13q31.3 | chr13:88,883,889-89,379,578 | 1 | VMRC-LCP | | |
| | RP11-79H7 | 13q31.3 | chr13:89,556,263-90,080,836 | 1 | VRAC-LCP | | |
| 8 | RP11-72J7 | 13q32.2 | chr13:97,465,157-97,495,769 | 1 | VMRC-LCD | EBI2 | 10 |
| | RP11-19J14 | 13q32.2 | chr13:97,851,595-97,851,757 | 1 | VMRC-LCD | | |
| | RP11-122A8 | 13q32.3 | chr13:98,471,182-98,644,517 | 1 | VMRC-LCD | | |
| 9 | RP11-134G22 | 20p12.1 | chr20:15,224,211-15,251,444 | 2 | SK-LC-3, KNS-62 | | 0 |
| | RP11-65G18 | 20p12.1 | chr20:15,271,770-15,375,903 | 1 | SK-LC-3 | | |
| | RP11-11O15 | 20p12.1 | chr20:15,567,963-15,728,758 | 1 | SK-LC-3 | | |

^{a}Based on UCSC Genome Browser, May 2004 Assembly.
^{b}Possible tumor suppressor genes located around BAC.

### (3) CONCLUSIONS

By screening using array CGH, a gene that is homozygously deleted in its genomic DNA, i.e., a gene that lacks genomic region in the non-small cell lung cancer cell line, was isolated. By detecting the existence of such gene, cancer can be diagnosed.

### Effects of the Invention

The present invention provides a novel method for diagnosing cancer comprising detecting the cancer-associated GMDS, ANKRD15, TEK, or EBI2 gene or the GMDS, ANKRD15, TEK, or EBI2 protein encoded by such gene. Analysis of such gene or gene product is very useful from the clinical point of view in terms of improvement in treatment or cancer prognosis based on individuality of cancer or from the viewpoint of fundamental cancer research. Also, assay of the mRNA expression level of the GMDS, ANKRD15, TEK, or EBI2 gene enables selection of a patient with non-small cell lung cancer.

1. A method for diagnosing cancer which comprises a step of detecting deletion of the GMDS, ANKRD15, TEK, or EBI2 gene in a test sample by using DNA containing all or part of said gene.

2. A method for diagnosing cancer which comprises a step of analyzing the GMDS, ANKRD15, TEK, or EB12 gene in a test sample by using DNA or RNA containing all or part of the GMDS, ANKRD15, TEK, or EBI2 gene.

3. The method of claim 2 wherein the analysis involves detection of mutation of the gene or detection of abnormal expression level of the gene.

4. A method for diagnosing cancer which comprises a step of analyzing GMDS, ANKRD15, TEK, or EBI2 protein in a test sample using an antibody against GMDS, ANKRD15, TEK, OR EBI2 protein or fragment thereof.

5. The method of claim 4 wherein the analysis involves detection of abnormal expression level of the protein.

6. The method of claim 1 wherein lung cancer is diagnosed.

7. The method of claim 2 wherein lung cancer is diagnosed.

8. The method of claim 4 wherein lung cancer is diagnosed.


SEQUENCE LISTING (10 sequences)

SEQ ID NO: 1 - DNA, 1698 bp, Homo sapiens
cccggccctccctgcacggcctcccgtgcgcccctgtcagactgtggcggccggtcgcgcggtgcgctctccctccctgcccgcagcctggagaggcgcttcgtgctgcacacccccgcgttcctgccggcaccgcgcctgccctctgccgcgctccgccctgccgccgaccgcacgcccgccgcgggacatggcacacgcaccggcacgctgccccagcgcccggggctccggggacggcgagatgggcaagcccaggaacgtggcgctcatcaccggtatcacaggccaggatggttcctacctggctgagttcctgctggagaaaggctatgaggtccatggaattgtacggcggtccagttcatttaatacgggtcgaattgagcatctgtataagaatccccaggctcacattgaaggaaacatgaagttgcactatggcgatctcactgacagtacctgccttgtgaagatcattaatgaagtaaagcccacagagatctacaaccttggagcccagagccacgtcaaaatttcctttgacctcgctgagtacactgcggacgttgacggagttggcactctacgacttctagatgcagttaagacttgtggccttatcaactctgtgaagttctaccaagcctcaacaagtgaactttatgggaaagtgcaggaaataccccagaaggagaccacccctttctatccccggtcaccctatggggcagcaaaactctatgcctattggattgtggtgaacttccgtgaggcgtataatctctttgcagtgaacggcattctcttcaatcatgagagtcccagaagaggagctaatttcgttactcgaaaaattagccggtcagtagctaagatttaccttggacaactggaatgtttcagtttgggaaatctggatgccaaacgagattggggccatgccaaggactatgtggaggctatgtggttgatgttgcagaatgatgagccggaggacttcgttatagctactggggaggtccatagtgtccgggaatttgtcgagaaatcattcttgcacattggaaaaaccattgtgtgggaaggaaagaatgaaaatgaagtgggcagatgtaaagagaccggcaaagttcacgtgactgtggatctcaagtactaccggccaactgaagtggactttctgcagggcgactgcaccaaagcgaaacagaagctgaactggaagccccgggtcgctttcgatgagctggtgagggagatggtgcacgccgacgtggagctcatgaggacaaaccccaatgcctgagcagcgcctcggagcccggcccgccctccggctacaatccccgcagagtctccggtgcagacgcgctgcggggatggggagcggcgtgccaatctgcgggtcccctgcggcccctgctgccgctgcgctgtcccggccgcaagagcggggccgccccgccgaggtttgtagcagccgggatgtgaccctccagggtttgggtcgctttgcgtttgtcgaagcctcctctgaatggctttgtgaaatcaagatgttttaatcacattcactttacttgaaattatgttgttacacaacaaattgtggggccttcaaattgtttttctcttttcatattaaaaatggtctttctgtgaactagcaaaaaaaaaaaaaaaaaaaaaaaaaaa

SEQ ID NO: 2 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 3 - DNA, 5083 bp, Homo sapiens
ggtccgcggcggagcgagcgagcggccggcaggttgggaggagcggccgaaggttgaatgcctttgagaacttgatgcataaaatttgcatgactcctcactcctttctggatctctcattggactcaagccagcatggctcacaccacaaaggttaacggcagtgcctcaggaaaagcaggtgatattctcagtggagaccaggacaaggaacagaaagacccttactttgtggagaccccctatggttatcaactagacttagatttcctcaaatatgtggatgacatacagaagggaaataccatcaaaagactgaacatccagaagaggcggaagccgtccgtgccatgcccagaacccaggaccacatctggtcagcaaggtatatggacttccactgaatccctctcatcctccaacagtgatgacaacaagcagtgccccaacttcctcatagccagaagtcaagttacatcaactccaatctcaaagccacctccccctctggagacctcactcccttttcttaccatcccagaaaatcgacagctgccacctccctcaccacaactcccaaagcataaccttcatgtcaccaagacactgatggagacccggagaagactggaacaggagagagccaccatgcagatgacaccgggtgagttcagaaggcccaggctggccagttttggaggcatgggcaccacaagctccctcccttcttttgtgggttctggaaaccacaatcctgccaagcaccagcttcagaatggataccaaggtaatggggattatggtagctatgccccagctgctcccaccacttcctccatggggagctccatccgccacagccccctgagctcagggatctccaccccagtgaccaacgtgagccccatgcacctgcagcacatccgcgagcagatggccattgctctgaaacgcctgaaggagctggaggagcaggtgcgaaccatccctgtgctccaggtaaagatctctgtcttgcaagaagagaaaaggcagttggtctcacagctgaaaaaccaaagggctgcatcccagatcaatgtctgtggtgtgaggaagcggtcctatagtgcggggaacgcctcccagctggaacagctctcccgggcccgaagaagtggcggggaattatacattgactatgaggaggaagaaatggagaccgtagaacagagcacgcagaggataaaggagttccggcaacttacagcagacatgcaagccctggagcagaagatccaggacagcagctgtgaggcctcctcagagctcagggagaatggagagtgccggtctgtggctgtgggtgccgaggagaacatgaacgacatcgtcgtgtaccacagaggctccaggtcctgtaaggatgcagctgtagggacacttgttgagatgagaaattgtggggtcagcgtgacagaggccatgcttggagtgatgactgaagctgacaaagaaattgagctgcaacagcagaccatagaatccttgaaggaaaagatctatcgcctagaagtacagcttagagaaaccacccatgaccgggagatgactaaactgaaacaagagctgcaggctgctggatcgaggaaaaaggttgacaaagccacgatggcccagccgcttgttttcagtaaggtggtggaggcagtggtgcagaccagagaccaaatggtcggcagtcacatggacctggtggacacgtgtgttgggacctccgtggaaacaaacagtgtaggcatctcctgccagcctgaatgtaagaataaagtcgtagggcctgagctgcctatgaattggtggattgttaaggagagggtggaaatgcatgaccgatgtgctgggaggtctgtggaaatgtgtgacaagagtgtgagtgtggaagtcagcgtctgcgaaacaggcagcaacacagaggagtctgtgaacgacctcacactcctcaagacaaacttgaatctcaaagaagtgcggtctatcggttgtggagattgttctgttgacgtgaccgtctgctctccaaaggagtgcgcctcccggggcgtgaacactgaggctgttagccaggtggaagctgccgtcatggcagtgcctcgtactgcagaccaggacactagcacagatttggaacaggtgcaccagttcaccaacaccgagacggccaccctcatagagtcctgcaccaacacttgtctaagcactttggacaagcagaccagcacccagactgtggagacgcggacagtagctgtaggagaaggccgtgtcaaggacatcaactcctccaccaagacgcggtccattggtgttggaacgttgctttctggccattctgggtttgacaggccatcagctgtgaagaccaaagagtcaggtgtggggcagataaatattaacgacaactatctggttggtctcaaaatgaggactatagcttgtgggccaccacagttgactgtggggctgacagccagcagaaggagcgtgggggttggggatgaccctgtaggggaatctctggagaacccccagcctcaagctccacttggaatgatgactggcctggatcactacattgagcgtatccagaagctgctggcagaacagcagacactgctggctgagaactacagtgaactggcagaagctttcggggaacctcactcacagatgggctccctcaactctcagctcatcagcaccctgtcgtctatcaactctgtcatgaaatctgcaagcactgaagagctgaggaaccctgacttccagaaaaccagtctgggtaaaatcacaggcaattatttgggatatacctgtaagtgtgggggccttcagtcaggaagtcccttaagctcccagacatcccagcctgagcaagaagtggggacctcagaaggaaagccaatcagcagcctggatgccttccccactcaggaaggtacgctgtctccagtgaacctgacagacgaccagatcgccgctggcctctatgcatgtacaaacaatgaaagtacactgaagtccatcatgaagaagaaagatggtaacaaagattcaaatggcgcaaaaaagaatcttcagtttgttggcattaatggagggtatgaaacaacttcaagtgatgattccagctcagatgaaagctcttcttccgagtcagatgacgagtgtgatgtcattgagtatcctcttgaagaagaggaggaggaggaggatgaagacactcggggaatggcagaagggcaccatgcagttaatattgaaggtttgaagtctgccagggtggaagatgaaatgcaggttcaagaatgtgaacctgagaaggtggaaatcagagagaggtatgaattaagtgaaaagatgttgtctgcatgcaacttactgaaaaatactataaatgaccccaaagctttgaccagcaaagatatgaggttctgtctgaacaccctccagcacgagtggttccgcgtgtccagtcagaagtcagccattccagccatggtgggggactacatagctgcttttgaggccatttccccagatgtcctccgctatgtcatcaacttggcagacggcaacggcaacacagccctccattacagcgtgtcccactccaacttcgagattgtgaagctgctgttagatgccgatgtgtgtaatgtggatcaccagaacaaggcaggctacacccccatcatgttggcggccctcgccgctgtggaagcagagaaggacatgcggattgtggaagaactcttcggctgtggggatgtgaatgccaaagctagtcaggcgggacagacggccctcatgctggcggtcagtcacggacggatagacatggtgaagggccttctggcctgtggggctgatgtcaacatccaggatgacgagggctccacggccctcatgtgtgccagcgagcacgggcacgtggagattgtcaagctgctgctggcccagcccggctgcaacggtcacctagaggacaacgatggcagcactgcgctctcaatcgccctggaagcaggacacaaggacatcgctgttcttctgtatgcccatgtcaactttgcaaaagcccagtctccgggcacccctaggcttggaaggaagacgtctcctggccccacccaccgaggttcatttgattgattgtatgcaaatagccctttatttacatgccactattaagctgctaattgttcctgttggggtgacagatactgaatgtatacgtattgtgcctgagctcaccagcaaacagaagcatcaagcccaggggtaaaggctgaagctttcacagtgcagagactgctagcctgggcacacacacctcctttctggccgtcttctgtgtagggcacactttaacccagtctctgttgctgttgagtctctgctccgttttgtacagtcacagggaattctgatctgaaggggcaccttctgttcactcccacaaagtggtgtctggttctcactgagacgttttaagatttttccacaaatatttatatgtactaaatgtggaaccattagaaagttcttccaaaatctcattccagcatagttttggatttttcttttgtcttattttaaaataaggaagtcgagatgactttgatcattggtaacttgggcctgggccagacaaagtataaaacttacaaaagaatattctcatttggtcttaactaggtagatgtaatatatgactttttataaaaagggtatctatatgaacttgacacagtattttcagcttttgtattccatactaaagccatgaagaactacacgtaacatcatcatttgtattaattgcacaactccaatgctaaaggttggattgtgttagaggaatcggctctgtatttgcctctagagaaacacagtgttctctttgtatttatggattcctttttaccgtgtcacatttactttggtcctctatgtatttaaatgtttgaagtgccttagactcttgccatattttcaaaataaaattccattaagctctaaaaaaaaaaaaaaaaaaaaa

SEQ ID NO: 4 - DNA, 5263 bp, Homo sapiens
agagcctctctgtagagatgcttaaagtgctagctttatttaaaaatcagctggcaaagcgggagtgaagaacacacattttcagaagataccggtttcctgttttcattaagaaaacagggaatgcggaacagctgagtgcggcgggggtggtgtcactgcagccggagggagacctcgccggtgaaagctcagcctatggcatccgagcgtggccggccaagtttacacgaatgttgaaacttctcctcacggggtgaggatcgagggcgcccgaggccgggtccggctgagctggagcgagctgtgcggggcagcgcgggctggcggggagcgcggcgggcgccacgtggaaaagcaggtgatattctcagtggagaccaggacaaggaacagaaagacccttactttgtggagaccccctatggttatcaactagacttagatttcctcaaatatgtggatgacatacagaagggaaataccatcaaaagactgaacatccagaagaggcggaagccgtccgtgccatgcccagaacccaggaccacatctggtcagcaaggtatatggacttccactgaatccctctcatcctccaacagtgatgacaacaagcagtgccccaacttcctcatagccagaagtcaagttacatcaactccaatctcaaagccacctccccctctggagacctcactcccttttcttaccatcccagaaaatcgacagctgccacctccctcaccacaactcccaaagcataaccttcatgtcaccaagacactgatggagacccggagaagactggaacaggagagagccaccatgcagatgacaccgggtgagttcagaaggcccaggctggccagttttggaggcatgggcaccacaagctccctcccttcttttgtgggttctggaaaccacaatcctgccaagcaccagcttcagaatggataccaaggtaatggggattatggtagctatgccccagctgctcccaccacttcctccatggggagctccatccgccacagccccctgagctcagggatctccaccccagtgaccaacgtgagccccatgcacctgcagcacatccgcgagcagatggccattgctctgaaacgcctgaaggagctggaggagcaggtgcgaaccatccctgtgctccaggtaaagatctctgtcttgcaagaagagaaaaggcagttggtctcacagctgaaaaaccaaagggctgcatcccagatcaatgtctgtggtgtgaggaagcggtcctatagtgcggggaacgcctcccagctggaacagctctcccgggcccgaagaagtggcggggaattatacattgactatgaggaggaagaaatggagaccgtagaacagagcacgcagaggataaaggagttccggcaacttacagcagacatgcaagccctggagcagaagatccaggacagcagctgtgaggcctcctcagagctcagggagaatggagagtgccggtctgtggctgtgggtgccgaggagaacatgaacgacatcgtcgtgtaccacagaggctccaggtcctgtaaggatgcagctgtagggacacttgttgagatgagaaattgtggggtcagcgtgacagaggccatgcttggagtgatgactgaagctgacaaagaaattgagctgcaacagcagaccatagaatccttgaaggaaaagatctatcgcctagaagtacagcttagagaaaccacccatgaccgggagatgactaaactgaaacaagagctgcaggctgctggatcgaggaaaaaggttgacaaagccacgatggcccagccgcttgttttcagtaaggtggtggaggcagtggtgcagaccagagaccaaatggtcggcagtcacatggacctggtggacacgtgtgttgggacctccgtggaaacaaacagtgtaggcatctcctgccagcctgaatgtaagaataaagtcgtagggcctgagctgcctatgaattggtggattgttaaggagagggtggaaatgcatgaccgatgtgctgggaggtctgtggaaatgtgtgacaagagtgtgagtgtggaagtcagcgtctgcgaaacaggcagcaacacagaggagtctgtgaacgacctcacactcctcaagacaaacttgaatctcaaagaagtgcggtctatcggttgtggagattgttctgttgacgtgaccgtctgctctccaaaggagtgcgcctcccggggcgtgaacactgaggctgttagccaggtggaagctgccgtcatggcagtgcctcgtactgcagaccaggacactagcacagatttggaacaggtgcaccagttcaccaacaccgagacggccaccctcatagagtcctgcaccaacacttgtctaagcactttggacaagcagaccagcacccagactgtggagacgcggacagtagctgtaggagaaggccgtgtcaaggacatcaactcctccaccaagacgcggtccattggtgttggaacgttgctttctggccattctgggtttgacaggccatcagctgtgaagaccaaagagtcaggtgtggggcagataaatattaacgacaactatctggttggtctcaaaatgaggactatagcttgtgggccaccacagttgactgtggggctgacagccagcagaaggagcgtgggggttggggatgaccctgtaggggaatctctggagaacccccagcctcaagctccacttggaatgatgactggcctggatcactacattgagcgtatccagaagctgctggcagaacagcagacactgctggctgagaactacagtgaactggcagaagctttcggggaacctcactcacagatgggctccctcaactctcagctcatcagcaccctgtcgtctatcaactctgtcatgaaatctgcaagcactgaagagctgaggaaccctgacttccagaaaaccagtctgggtaaaatcacaggcaattatttgggatatacctgtaagtgtgggggccttcagtcaggaagtcccttaagctcccagacatcccagcctgagcaagaagtggggacctcagaaggaaagccaatcagcagcctggatgccttccccactcaggaaggtacgctgtctccagtgaacctgacagacgaccagatcgccgctggcctctatgcatgtacaaacaatgaaagtacactgaagtccatcatgaagaagaaagatggtaacaaagattcaaatggcgcaaaaaagaatcttcagtttgttggcattaatggagggtatgaaacaacttcaagtgatgattccagctcagatgaaagctcttcttccgagtcagatgacgagtgtgatgtcattgagtatcctcttgaagaagaggaggaggaggaggatgaagacactcggggaatggcagaagggcaccatgcagttaatattgaaggtttgaagtctgccagggtggaagatgaaatgcaggttcaagaatgtgaacctgagaaggtggaaatcagagagaggtatgaattaagtgaaaagatgttgtctgcatgcaacttactgaaaaatactataaatgaccccaaagctttgaccagcaaagatatgaggttctgtctgaacaccctccagcacgagtggttccgcgtgtccagtcagaagtcagccattccagccatggtgggggactacatagctgcttttgaggccatttccccagatgtcctccgctatgtcatcaacttggcagacggcaacggcaacacagccctccattacagcgtgtcccactccaacttcgagattgtgaagctgctgttagatgccgatgtgtgtaatgtggatcaccagaacaaggcaggctacacccccatcatgttggcggccctcgccgctgtggaagcagagaaggacatgcggattgtggaagaactcttcggctgtggggatgtgaatgccaaagctagtcaggcgggacagacggccctcatgctggcggtcagtcacggacggatagacatggtgaagggccttctggcctgtggggctgatgtcaacatccaggatgacgagggctccacggccctcatgtgtgccagcgagcacgggcacgtggagattgtcaagctgctgctggcccagcccggctgcaacggtcacctagaggacaacgatggcagcactgcgctctcaatcgccctggaagcaggacacaaggacatcgctgttcttctgtatgcccatgtcaactttgcaaaagcccagtctccgggcacccctaggcttggaaggaagacgtctcctggccccacccaccgaggttcatttgattgattgtatgcaaatagccctttatttacatgccactattaagctgctaattgttcctgttggggtgacagatactgaatgtatacgtattgtgcctgagctcaccagcaaacagaagcatcaagcccaggggtaaaggctgaagctttcacagtgcagagactgctagcctgggcacacacacctcctttctggccgtcttctgtgtagggcacactttaacccagtctctgttgctgttgagtctctgctccgttttgtacagtcacagggaattctgatctgaaggggcaccttctgttcactcccacaaagtggtgtctggttctcactgagacgttttaagatttttccacaaatatttatatgtactaaatgtggaaccattagaaagttcttccaaaatctcattccagcatagttttggatttttcttttgtcttattttaaaataaggaagtcgagatgactttgatcattggtaacttgggcctgggccagacaaagtataaaacttacaaaagaatattctcatttggtcttaactaggtagatgtaatatatgactttttataaaaagggtatctatatgaacttgacacagtattttcagcttttgtattccatactaaagccatgaagaactacacgtaacatcatcatttgtattaattgcacaactccaatgctaaaggttggattgtgttagaggaatcggctctgtatttgcctctagagaaacacagtgttctctttgtatttatggattcctttttaccgtgtcacatttactttggtcctctatgtatttaaatgtttgaagtgccttagactcttgccatattttcaaaataaaattccattaagctctaaaaaaaaaaaaaaaaaaaaa

SEQ ID NO: 5 - protein, 1352 aa, Homo sapiens
MAHTTKVNGSASGKAGDILSGDQDKEQKDPYFVETPYGYQLDLDFLKYVDDIQKGNTIKRLNIQKRRKPSVPCPEPRTTSGQQGIWTSTESLSSSNSDDNKQCPNFLIARSQVTSTPISKPPPPLETSLPFLTIPENRQLPPPSPQLPKHNLHVTKTLMETRRRLEQERATMQMTPGEFRRPRLASFGGMGTTSSLPSFVGSGNHNPAKHQLQNGYQGNGDYGSYAPAAPTTSSMGSSIRHSPLSSGISTPVTNVSPMHLQHIREQMAIALKRLKELEEQVRTIPVLQVKISVLQEEKRQLVSQLKNQRAASQINVCGVRKRSYSAGNASQLEQLSRARRSGGELYIDYEEEEMETVEQSTQRIKEFRQLTADMQALEQKIQDSSCEASSELRENGECRSVAVGAEENMNDIVVYHRGSRSCKDAAVGTLVEMRNCGVSVTEAMLGVMTEADKEIELQQQTIESLKEKIYRLEVQLRETTHDREMTKLKQELQAAGSRKKVDKATMAQPLVFSKVVEAVVQTRDQMVGSHMDLVDTCVGTSVETNSVGISCQPECKNKVVGPELPMNWWIVKERVEMHDRCAGRSVEMCDKSVSVEVSVCETGSNTEESVNDLTLLKTNLNLKEVRSIGCGDCSVDVTVCSPKECASRGVNTEAVSQVEAAVMAVPRTADQDTSTDLEQVHQFTNTETATLIESCTNTCLSTLDKQTSTQTVETRTVAVGEGRVKDINSSTKTRSIGVGTLLSGHSGFDRPSAVKTKESGVGQININDNYLVGLKMRTIACGPPQLTVGLTASRRSVGVGDDPVGESLENPQPQAPLGMMTGLDHYIERIQKLLAEQQTLLAENYSELAEAFGEPHSQMGSLNSQLISTLSSINSVMKSASTEELRNPDFQKTSLGKITGNYLGYTCKCGGLQSGSPLSSQTSQPEQEVGTSEGKPISSLDAFPTQEGTLSPVNLTDDQIAAGLYACTNNESTLKSIMKKKDGNKDSNGAKKNLQFVGINGGYETTSSDDSSSDESSSSESDDECDVIEYPLEEEEEEEDEDTRGMAEGHHAVNIEGLKSARVEDEMQVQECEPEKVEIRERYELSEKMLSACNLLKNTINDPKALTSKDMRFCLNTLQHEWFRVSSQKSAIPAMVGDYIAAFEAISPDVLRYVINLADGNGNTALHYSVSHSNFEIVKLLLDADVCNVDHQNKAGYTPIMLAALAAVEAEKDMRIVEELFGCGDVNAKASQAGQTALMLAVSHGRIDMVKGLLACGADVNIQDDEGSTALMCASEHGHVEIVKLLLAQPGCNGHLEDNDGSTALSIALEAGHKDIAVLLYAHVNFAKAQSPGTPRLGRKTSPGPTHRGSFD

SEQ ID NO: 6 - protein, 1194 aa, Homo sapiens
METRRRLEQERATMQMTPGEFRRPRLASFGGMGTTSSLPSFVGSGNHNPAKHQLQNGYQGNGDYGSYAPAAPTTSSMGSSIRHSPLSSGISTPVTNVSPMHLQHIREQMAIALKRLKELEEQVRTIPVLQVKISVLQEEKRQLVSQLKNQRAASQINVCGVRKRSYSAGNASQLEQLSRARRSGGELYIDYEEEEMETVEQSTQRIKEFRQLTADMQALEQKIQDSSCEASSELRENGECRSVAVGAEENMNDIVVYHRGSRSCKDAAVGTLVEMRNCGVSVTEAMLGVMTEADKEIELQQQTIESLKEKIYRLEVQLRETTHDREMTKLKQELQAAGSRKKVDKATMAQPLVFSKVVEAVVQTRDQMVGSHMDLVDTCVGTSVETNSVGISCQPECKNKVVGPELPMNWWIVKERVEMHDRCAGRSVEMCDKSVSVEVSVCETGSNTEESVNDLTLLKTNLNLKEVRSIGCGDCSVDVTVCSPKECASRGVNTEAVSQVEAAVMAVPRTADQDTSTDLEQVHQFTNTETATLIESCTNTCLSTLDKQTSTQTVETRTVAVGEGRVKDINSSTKTRSIGVGTLLSGHSGFDRPSAVKTKESGVGQININDNYLVGLKMRTIACGPPQLTVGLTASRRSVGVGDDPVGESLENPQPQAPLGMMTGLDHYIERIQKLLAEQQTLLAENYSELAEAFGEPHSQMGSLNSQLISTLSSINSVMKSASTEELRNPDFQKTSLGKITGNYLGYTCKCGGLQSGSPLSSQTSQPEQEVGTSEGKPISSLDAFPTQEGTLSPVNLTDDQIAAGLYACTNNESTLKSIMKKKDGNKDSNGAKKNLQFVGINGGYETTSSDDSSSDESSSSESDDECDVIEYPLEEEEEEEDEDTRGMAEGHHAVNIEGLKSARVEDEMQVQECEPEKVEIRERYELSEKMLSACNLLKNTINDPKALTSKDMRFCLNTLQHEWFRVSSQKSAIPAMVGDYIAAFEAISPDVLRYVINLADGNGNTALHYSVSHSNFEIVKLLLDADVCNVDHQNKAGYTPIMLAALAAVEAEKDMRIVEELFGCGDVNAKASQAGQTALMLAVSHGRIDMVKGLLACGADVNIQDDEGSTALMCASEHGHVEIVKLLLAQPGCNGHLEDNDGSTALSIALEAGHKDIAVLLYAHVNFAKAQSPGTPRLGRKTSPGPTHRGSFD

SEQ ID NO: 7 - DNA, 4138 bp, Homo sapiens
cttctgtgctgttccttcttgcctctaacttgtaaacaagacgtactaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcggctgtgatttcttggacaatattggatggctattctatttcttctattactatccgttacaaggttcaaggcaagaatgaagaccagcacgttgatgtgaagataaagaatgccaccatcattcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtacgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacaactgctgagaaaacatgcctctgccaaaggatgtgatatataagtgtacatatgtgctggaattctaacaagtcataggttaatatttaagacactgaaaaatctaagtgatataaatcagattcttctctctcattttatccctcacctgtagcatgccagtcccgtttcatttagtcatgtgaccactctgtcttgtgtttccacagcctgcaagttcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcaaaaaaaaaaaa

SEQ ID NO: 8 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATIIQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 9 - DNA, 1746 bp, Homo sapiens
tgcacagcctctcttaaaacaacatgccacagttaaaacttacttcagtttggacaactactcacagctactacacagagacccgaacgagtcactgatatacacctggaccaccaccaatggatatacaaatggcaaacaattttactccgccctctgcaactcctcagggaaatgactgtgacctctatgcacatcacagcacggccaggatagtaatgcctctgcattacagcctcgtcttcatcattgggctcgtgggaaacttactagccttggtcgtcattgttcaaaacaggaaaaaaatcaactctaccaccctctattcaacaaatttggtgatttctgatatactttttaccaccgctttgcctacacgaatagcctactatgcaatgggctttgactggagaatcggagatgccttgtgtaggataactgcgctagtgttttacatcaacacatatgcaggtgtgaactttatgacctgcctgagtattgaccgcttcattgctgtggtgcaccctctacgctacaacaagataaaaaggattgaacatgcaaaaggcgtgtgcatatttgtctggattctagtatttgctcagacactcccactcctcatcaaccctatgtcaaagcaggaggctgaaaggattacatgcatggagtatccaaactttgaagaaactaaatctcttccctggattctgcttggggcatgtttcataggatatgtacttccacttataatcattctcatctgctattctcagatctgctgcaaactcttcagaactgccaaacaaaacccactcactgagaaatctggtgtaaacaaaaaggctctcaacacaattattcttattattgttgtgtttgttctctgtttcacaccttaccatgttgcaattattcaacatatgattaagaagcttcgtttctctaatttcctggaatgtagccaaagacattcgttccagatttctctgcactttacagtatgcctgatgaacttcaattgctgcatggacccttttatctacttctttgcatgtaaagggtataagagaaaggttatgaggatgctgaaacggcaagtcagtgtatcgatttctagtgctgtgaagtcagcccctgaagaaaattcacgtgaaatgacagaaacgcagatgatgatacattccaagtcttcaaatggaaagtgaaatggattgtattttggtttatagtgacgtaaactgtatgacaaactttgcaggacttcccttataaagcaaaataattgttcagcttccaattagtattcttttatatttctttcattgggcactttcccatctccaactcggaagtaagcccaagagaacaacataaagcaaacaacataaagcacaataaaaatgcaaataaatatttcatttttatttgtaaacgaatacaccaaaaggaggcgctcttaataactcccaatgtaaaaagttttgttttaataaaaaatttaattattatttcttgccaacaaatggctagaaaggactgaatagattatatattgccagatgttaatactgtaacatactttttaaataacatatttcttaaatccaaatttctctcaatgttagatttaattccctcaataacaccaatgttttgttttgtttcgttctgggtcataaaactttgttaaggaactcttttggaataaagagcaggatgctgcaaagttaaaaaaaaaaaaaaaaa

SEQ ID NO: 10 - protein, 361 aa, Homo sapiens [sequence omitted]